(19) United States
(12) Patent Application Publication
(10) Pub. No.: US 2010/0081904 A1
(43) Pub. Date: Apr. 1, 2010

(54) Device And Method For Securing A Medical Sensor to An Infant's Head

(75) Inventor: Casey V. Medina, Westminister, CO (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/241,267

(22) Filed: Sep. 30, 2008

(51) Int. Cl. A61B 5/00

(52) U.S. Cl. 600/340; 600/323; 600/344

(57) ABSTRACT

The present disclosure generally relates to a device and method for securing a sensor to a wearer's head. According to embodiments, a headcovering, such as a stocking cap, includes an integral headband. The headband may have a generally inelastic segment capable of being placed about the wearer's head and a generally elastic portion capable of fastening the headband in a secure fashion. The headband may include an indicator that facilitates the determination of whether the headband has been fastened at an appropriate tension about the wearer's head. The headband may also include dimensional markings to facilitate the measurement of the circumference of the wearer's head.

### BACKGROUND

The present disclosure generally relates to medical sensors and in particular, to hat-based pulse oximeter sensors.

This section is intended to introduce the reader to aspects of the at that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Many types of medical sensors, such as optical sensors, are used to measure physiological characteristics of a patient. Typically, an optical sensor provides emitted light which is then scattered through a portion of a patient's tissue and detected. Various characteristics of a patient can be determined from analyzing such light, such as oxygen saturation, pulse rate, tissue bilibrubin, etc.

Pulse oximetry is typically used to measure various blood flow characteristics including, but not limited to, the blood-oxygen saturation of hemoglobin in arterial blood, the volume of individual blood pulsations supplying the tissue, and the rate of blood pulsations corresponding to each heartbeat of a patient. Measurement of these characteristics has been accomplished by use of a non-invasive sensor which scatters light through a portion of the patient's tissue where blood perfuses the tissue, and photoelectrically senses the absorption of light in such tissue. The amount of light absorbed and/or scattered is then used to calculate the amount of blood constituent being measured.

The light transmitted through the tissue is selected to be of one or more wavelengths that are absorbed by the blood in an amount representative of the amount of the blood constituent present in the blood. The amount of transmitted light scattered through and/or absorbed by the tissue will vary in accordance with the changing amount of blood constituent in the tissue. For measuring blood oxygen level, such sensors have typically been provided with a light source that is adapted to generate light of at least two different wavelengths, in accordance with known techniques for measuring blood oxygen saturation.

Known non-invasive sensors include devices that are secured to a portion of the body, such as a finger, an ear, or the scalp. In animals and humans, the tissue of these body portions is perfused with blood and the tissue surface is readily accessible to the sensor. More particularly, certain types of oximeter sensors are applied to a patient's forehead. For example, an oximeter sensor attached to the inside of a stocking hat provides an easy means of placing, retaining, and locating the sensor on an infant's forehead. Such hats should preferably be the correct size for the infant's head to ensure that the sensor is in contact with the tissue and applying the optimal pressure to the infant's forehead. Indeed, measurement accuracy may diminish if the hat is too tight, due to diminished blood volume and perfusion of underlying tissue, or if the hat is too loose, due to venous pulsations and/or less than optimal sensor contact.

In addition to the various measurements provided by oximeter sensors, head circumference measurements are often taken by caregivers to determine an infant's developmental progress and to detect abnormal brain and skull growth. Presently, any head covering that the infant may have, whether it be a normal stocking cap or an oximetry sensor that is coupled to a stocking cap, must be removed so that such a measurement can be taken. The removal of the stocking cap not only can affect the infant's ability to maintain its temperature, but in the case of the removal of a stocking cap having an oximetry sensor, also the ability to continue taking oximetry measurements.

### BRIEF DESCRIPTION OF THE DRAWINGS

FIG. 1 is a drawing of a stocking hat, in accordance with an embodiment of the present disclosure.

FIG. 2 is a drawing of an oximeter sensor used with the stocking hat of FIG. 1.

FIG. 3 is a cutaway view of the stocking hat of FIG. 1 with the oximeter sensor of FIG. 2 attached.

FIG. 4 is a patient monitoring system coupled to a multi-parameter patient monitor and the oximeter sensor of FIG. 3.

### DETAILED DESCRIPTION

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The presently disclosed embodiments are directed towards adjusting a stocking hat containing a reflectance-type oximeter sensor to fit different head sizes and using the hat to measure the circumference of a user's head. With regard to the location of the sensor on the patient's forehead, the sensor may be located on the lower forehead region, above the eyebrow, with the sensor optics (emitter and detector) located above and predominantly lateral to or centered over the iris. The oximeter sensor can be attached to the inside of a stocking hat for use on an infant, for example. Coupling the sensor with a stocking hat allows easy placement of the sensor on the infant's forehead while applying a predictable pressure on the sensor. As discussed below, the stocking hat's ability to adapt to different head sizes further ensures that the sensor applies optimal pressure to the infant's forehead. Further, dimensional markings on the stocking hat allow the infant's head circumference to be measured without disrupting the placement of the sensor. Thus, a stocking hat with an attached oximeter sensor can be used on patients to measure heart rate, oxygen saturation, head circumference, and other parameters.

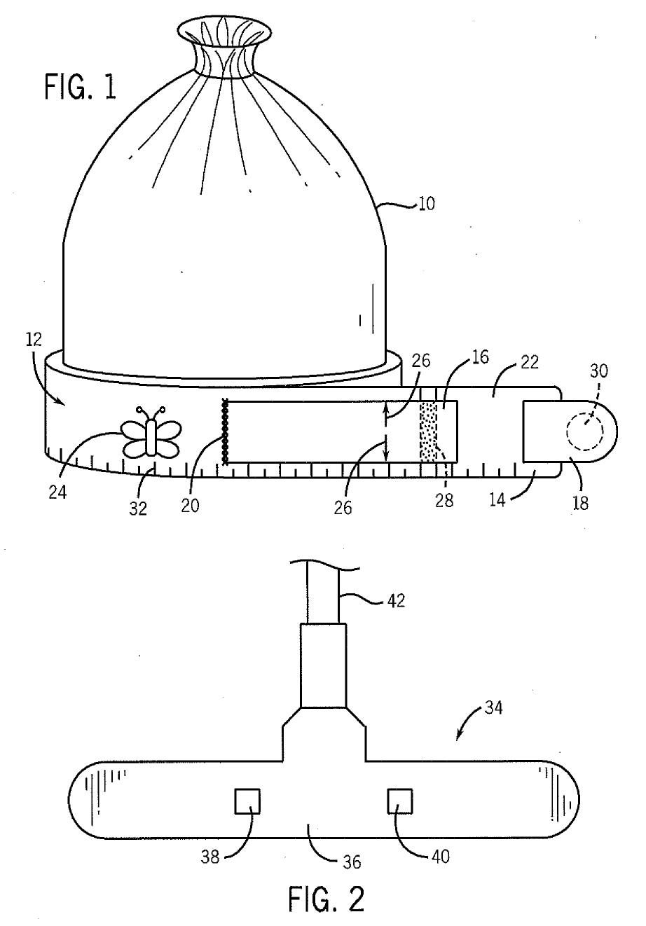

FIG. 1 is a drawing of a stocking hat in accordance with an embodiment. In this example, the stocking hat 10 has an adjustable headband 12. As illustrated, the headband 12 may be integral to the stocking hat 10, such that it is inserted into a pocket formed around the periphery of the stocking hat 10. The headband 12 may include a low-stretch, i.e., generally inelastic, segment 14 sized to fit around an infant's head, and a generally elastic band 16 that may be coupled to the low-stretch segment 14. The generally elastic band 16 may be elastic along substantially its entire length, or it may include an elastic portion and an inelastic portion. In this embodiment, the elastic band 16 has a loose end 18 and an attached end 20, where the elastic band 16 is attached at its attached end 20 with the low-stretch segment 14. The elastic band 16 is threaded through a guide band 22 of the low-stretch segment 14, which functions to prevent slippage of the elastic band 16. The elastic band 16 also may include tension arrows 26 that align with a tension indicator zone 28 on the low-stretch segment 14 when the elastic band 16 is in a stretched state. In this embodiment, the opposite face of the loose end 18 of the elastic band 16 has hook and loop fasteners 30 which may couple to the low-stretch segment 14 and/or the stocking hat 10 to affix to the headband 12 around the infant's head and maintain the headband 12 at the desired tension. Hence, when the low-stretch segment 14 has been placed about an infant's head and secured in the proper range with the tension arrows 26 aligned in the tension indicator zone 28, the stocking hat 10 should be adequately secured to the infant's head in a manner that will facilitate proper sensor readings from the sensor described below.

Also, the bottom of the low-stretch segment 14 may have dimensional markings 32 which allow the infant's head circumference to be measured without removing the stocking hat 10. For example, the dimensional markings 32 may be segmented in inches at 1/16^{th} intervals, or in centimeters at millimeter intervals. Since the dimensional markings 32 are on the low-stretch segment 14, the measurement thus provided should remain accurate even after the headband 12 has been secured to the infant's head.

FIG. 2 is a drawing of an oximeter sensor. A sensor 34, as discussed herein, may be configured for reflective type sensing. Furthermore, the sensor 34 may include various structural and functional features designed to facilitate its use. Examples of such sensor and its use and construction may be found in U.S. Pat. No. 7,047,056, which issued on May 16, 2006, as well as U.S. application Ser. No. 11/494,357 titled “Hat-Based Oximeter Sensor,” and filed on Jul. 26, 2006, which are both herein incorporated by reference in their entirety for all purposes. In the illustrated embodiment, the sensor 34 includes a flexible circuit 36, on which an emitter 38 and a detector 40 may be mounted. The flexible circuit 36 may be used to transmit signals to the emitter 38 and from the detector 40 via a cable 42. The emitter 38 may be one or more light emitting diodes adapted to transmit one or more wavelengths of light, such as a red to infrared range, and the detector 40 may be a photodetector, such as a silicon photodiode package, selected to receive light in the range emitted from the emitter 38. In an embodiment, the sensor 34 is coupled to the cable 42 that may be used to transmit electrical and/or optical signals to and from the emitter 38 and the detector 40. The cable 42 may be permanently or removably coupled to the sensor 34. The removable coupling of the cable 42 may be utilized in situations where the sensor 34 is disposable, e.g., where a sensor is disposed of after being used on a patient.

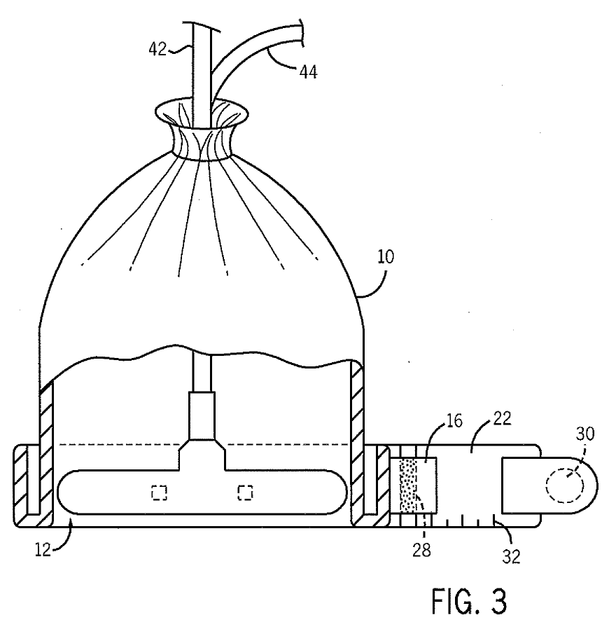

With regard to the location of the sensor 34 on a patients forehead, the sensor 34 may be situated on the lower forehead region, above the eyebrow, with the sensor optical devices located above and predominantly lateral to or centered over the iris. In the depicted embodiment of FIG. 3, the sensor 34 may be attached to the inside of the headband 12 and/or of the stocking hat 10. The low-stretch segment 14 or the stocking hat 10 also may have an indicia 24 (see FIG. 1) corresponding to the location of the underlying sensor 34. This facilitates proper placement of the stocking hat 10, and thus the sensor 34, on the infant's head. Hence, coupling the sensor 34 with a stocking hat 10 allows easy placement of the sensor 34 on the infant's forehead while applying a predictable pressure on the sensor 34.

In another embodiment, the top opening of the stocking hat 10 may provide an outlet for an intravenous tube 44 inserted into the patient. For infants in particular, it is not uncommon for tubes to be inserted in or near their heads. Since an infant's movement could potentially disturb an intravenous tube so placed, it may be advantageous to route the tube 44 through the top opening of the stocking hat 10 to minimize the possibility of such a disruption.

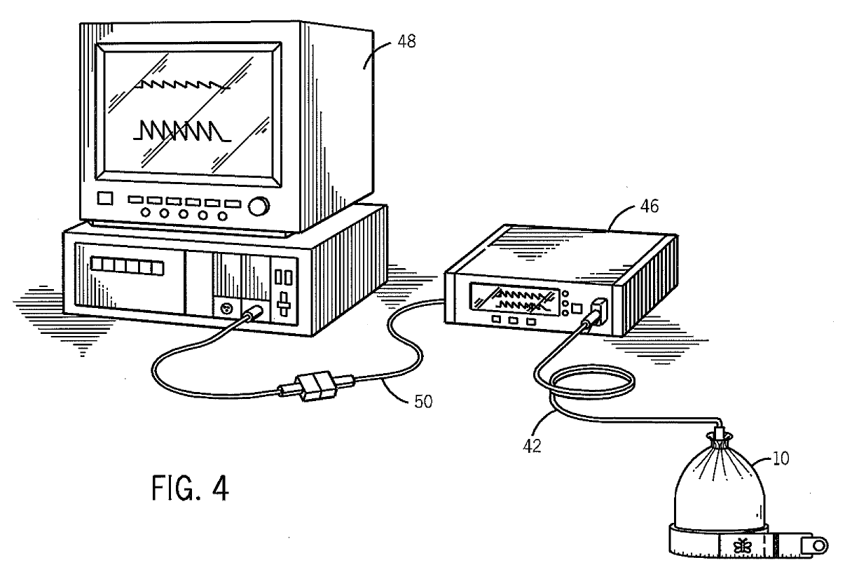

It should be appreciated that a stocking hat 10 with an attached oximeter sensor 34 is designed for use with a patient monitoring system. For example, referring now to FIG. 4, the stocking hat 10 as depicted in FIG. 3 may be used in conjunction with a patient monitor 46. In an embodiment, a cable 42 connects the sensor 34 to the patient monitor 46. The sensor 34 and/or cable 42 may include or incorporate one or more integrated circuit or electrical devices, such as a memory processor chip, that may facilitate or enhance communication between the sensor 34 and the patient monitor 46. Similarly, the cable 42 may be an adaptor cable, with or without an integrated circuit or electrical device, for facilitating communication between the sensor 34 and various types of monitors, including different versions of the patient monitor 46 or other physiological monitors. In other embodiments, the sensor 34 and the patient monitor 46 may communicate via wireless means, such as using radio frequency, infrared or optical signals. In such embodiments, a transmission device may be connected to sensor 34 to facilitate wireless transmission between sensor 34 and patient monitor 46. The cable 42 (or a corresponding wireless connection) may typically be used to transmit control or timing signals from the patient monitor 46 to the sensor 34 and/or to transmit acquired data from the sensor 34 to the patient monitor 46. In other embodiments, the cable 42 may be an optical fiber that enables optical signals to be transmitted between the patient monitor 46 and the sensor 34.

In one embodiment, the patient monitor 46 may be a suitable pulse oximeter, such as those available from Nellcor Puritan Bennett L.L.C. In other embodiments, the patient monitor 46 may be a monitor suitable for measuring tissue water fractions, or other body fluid related metrics, using spectrophotometric or other techniques. Furthermore, the patient monitor 46 may be a multipurpose monitor suitable for performing pulse oximetry and measurement of tissue water fraction, or other combinations of physiological and/or biochemical monitoring processes, using data acquired via the sensor 34 and/or other sensors. Moreover, to upgrade conventional monitoring functions provided by the system, the patient monitor 46 may be coupled to a multi-parameter patient monitor 48 via a monitor cable 50 connected to a sensor input port and/or a cable connected to a digital communication port.

In summary, the ability of the stocking hat 10 to adapt to different head sizes helps to ensure that the sensor 34 applies optimal pressure to the infant's forehead. Further, dimensional markings 32 on the stocking hat 10 allow the infant's head circumference to be measured without removing the stocking hat 10 or disrupting the placement of the sensor 34. Indeed, a stocking hat 10 with an attached oximeter sensor 34 as depicted in FIG. 3 can be used on patients in order to measure heart rate, oxygen saturation, head circumference, and/or other parameters.

While the disclosure may be susceptible to various modifications and alternative forms, specific embodiments have been shown by way of example in the drawings and have been described in detail herein. However, it should be understood that the disclosure is not intended to be limited to the particular forms provided. Rather, the disclosure is to cover all modifications, equivalents, and alternatives falling within the spirit and scope of the disclosure as defined by the following appended claims. Indeed) the present disclosed methods may not only be applied to transmission type sensors for use in pulse oximetry, but also to other sensor designs.

What is claimed is:

1. A sensor comprising:
a headcovering having a generally integral headband, the headband being capable of adjustably securing the headcovering to a wearer's head; and
a sensor disposed on the headcovering, the sensor being capable of communicatively coupling to a wearer's head.

2. The sensor of claim 1, wherein the headcovering comprises a neonatal stocking cap.

3. The sensor of claim 1, wherein the integral headband comprises:
a generally inelastic segment capable of being placed around a wearer's head; and
a generally elastic segment coupled to the generally inelastic segments the generally elastic segment having a fastener to secure the integral headband to a wearer's head.

4. The sensor of claim 3, wherein the fastener comprises a hook fastener capable of coupling to at least one of the headcoverirng or the generally inelastic segment.

5. The sensor of claim 1, wherein the integral headband comprises at least one indicator capable of indicating whether the integral headband has been secured to a wearer's head at an appropriate tension.

6. The sensor of claim 1, wherein the integral headband comprises dimensional markings capable of facilitating measurement of a circumference of a wearer's head.

7. The sensor of claim 1, wherein the sensor comprises a pulse oximetry sensor.

8. A device for measuring a circumference of an infant's head, the device comprising:
a headcovering capable of being placed on an infant's head, the headcovering comprising dimensional markings capable of facilitating measurement of a circumference of the infant's head without removing the headcovering from the infant's head.

9. The device of claim 8, wherein the headcovering comprises a neonatal stocking cap.

10. The device of claim 8, wherein the headcovering comprises an integral headband that includes a generally inelastic segment capable of being placed around the infant's head, wherein the dimensional markings are disposed on the generally inelastic segment.

11. The device of claim 10, wherein the integral headband comprises a generally elastic segment coupled to the generally inelastic segment, the generally elastic segment having a fastener to secure the integral headband to the infant's head.

12. The device of claim 11, wherein the fastener comprises a hook fastener capable of coupling to at least one of the headcoverings or the generally inelastic segment.

13. The device of claim 10, wherein the integral headband comprises at least one indicator capable of indicating whether the integral headband has been secured to the infant's head at an appropriate tension.

14. The device of claim 8, further comprising a sensor.

15. A pulse oximetry system comprising:
a pulse oximetry monitor; and
a pulse oximetry sensor operatively coupled to the pulse oximetry monitor, the pulse oximetry sensor comprising:
a headcovering having an integral headband, the headband being capable of adjustably securing the headcovering to a wearer's head; and
a light emitting and detecting sensor disposed on the headcovering, the light emitting and detecting sensor being capable of transmitting light into tissue of a wearer's head and receiving light from the tissue.

16. The system of claim 15, wherein the headcovering comprises a neonatal stocking cap.

17. The system of claim 15, wherein the integral headband comprises:
a generally inelastic segment capable of being placed around a wearer's head; and
a generally elastic segment coupled to the generally inelastic segment, the generally elastic segment having a fastener to secure the integral headband to a wearer's head.

18. The system of claim 17, wherein the fastener comprises a hook fastener capable of coupling to at least one of the headcoverings or the generally inelastic segment.

19. The system of claim 17, wherein the integral headband comprises at least one indicator capable of indicating whether the integral headband has been secured to a wearer's head at an appropriate tension.

20. The system of claim 17, wherein the integral headband comprises dimensional markings capable of facilitating measurement of a circumference of a wearer's head.

21. The system of claim 20, wherein the dimensional markings are disposed on a generally inelastic segment of the integral headband.
